(19) United States
(12) Patent Application Publication
(10) Pub. No.: US 2024/0024124 A1
(43) Pub. Date: Jan. 25, 2024

(54) INTERVERTEBRAL CAGE

(71) Applicant: Byeong Cheol Rim, Sejong (KR)

(72) Inventor: Byeong Cheol Rim, Sejong (KR)

(21) Appl. No.: 18/040,224

(22) PCT Filed: Dec. 2, 2020
(86) PCT No.: PCT/KR2020/017429

§ 371 Date: Feb. 1, 2023

(30) Foreign Application Priority Data
Aug. 4, 2020 (KR) 10-2020-0097172

(51) Int. Cl. A61F 2/44

(52) U.S. Cl. CPC A61F 2/4465; A61F 2002/448; A61F 2002/3065

(57) ABSTRACT

Intervertebral cage inserted between vertebrae disclosed. The intervertebral cage may include a first rotatable body, a second rotatable body, and ball joints, disposed on a lateral surface of the first rotatable body and a lateral surface of the second rotatable body facing the first rotatable body, and configured for coupling the first rotatable body rotatably to the second rotatable body and defining a rotation path through which the first rotatable body rotates toward the second rotatable body.

### FIELD

The present invention relates to an intervertebral cage inserted between vertebrae.

### RELATED ART

The spine consists of a plurality of vertebrae and intervertebral discs located between the vertebrae. The intervertebral disc functions to absorb and disperse shocks applied to the human body. When these intervertebral discs are damaged or displaced from their original positions, diseases such as spinal stenosis or disc herniation occur.

An intervertebral cage, which is intended to treat such spine-related diseases, is seated between the vertebrae and functions to maintain a distance between the vertebrae. In general, the intervertebral cage is manufactured in a straight or curved shape, and is inserted into the body by an insertion tool. In the case of a straight intervertebral cage, insertion is easy, but skill of a medical professional is required to place it in an appropriate position within the vertebrae. In the case of a curved intervertebral cage, it is difficult to insert it avoiding nerves or muscles due to its shape.

### SUMMARY

According to one aspect of the present invention, there is provide an intervertebral cage inserted between vertebrae. The intervertebral cage may include a first rotatable body, a second rotatable body, and ball joints, disposed on a lateral surface of the first rotatable body and a lateral surface of the second rotatable body facing the first rotatable body, and configured for coupling the first rotatable body rotatably to the second rotatable body and defining a rotation path through which the first rotatable body rotates toward the second rotatable body.

In one embodiment, the ball joints may include a ball, a connecting rod configured for connecting the ball and the lateral surface of the first rotatable body and a ball housing coupled to the lateral surface of the second rotatable body and configured for accommodating the ball.

In one embodiment, the ball housing may include a ball accommodating space, configured for accommodating the ball, a ball insertion hole formed to extend from a surface of the ball housing facing the lateral surface of the second rotatable body to the ball accommodation space and having a smaller diameter than that of the ball, a plurality of elastic grooves extending from the ball insertion hole toward the lateral surface of the second rotatable body, a ball accommodating space formed to extend from the ball insertion hole to the inside, and a guide groove extending from the ball insertion hole toward the lateral surface of the second rotatable body to define the rotation path along which the connecting rod moves when the ball rotates about its center as an axis.

In one embodiment, at least a portion of the ball accommodating space may be formed to extend inwardly from the lateral surface of the second rotatable body.

In one embodiment, a bottom surface of each of the first rotatable body and the second rotatable body may be flat, and the bottom surfaces of the center body, the first rotatable body, and the second rotatable body may be arranged in parallel before the first rotatable body and the second rotatable body rotate.

In one embodiment, a top surface of each of the first rotatable body and the second rotatable body may be a curved surface, and top surfaces of the first rotatable body and the second rotatable body may be arranged in an arc shape when the first rotating body rotates.

In one embodiment, the lateral surface of the first rotatable body and the lateral surface of the second rotatable body may be convex curved surfaces.

In one embodiment, the lateral surface of the first rotatable body and the lateral surface of the second rotatable body may be inclined with respect to the respective bottom surfaces.

In one embodiment, at least one of the first rotatable body and the second rotatable body comprises a bone powder receiving hole penetrating the inside.

An intervertebral cage according to embodiments of the present invention has a shape close to a substantially straight line when inserted, and can be deformed into a shape along the medial wall of a vertebra when seated after insertion. Due to this, when inserting the intervertebral cage, the incision site can be minimized, and subsequent sagging for adjusting the position of the intervertebral cage after insertion becomes unnecessary.

### BRIEF DESCRIPTION OF THE DRAWINGS

Hereinafter, embodiments of the invention will be described with reference to the accompanying drawings. For the purpose of easy understanding of the invention, the same elements will be referred to by the same reference signs. Configurations illustrated in the drawings are examples for describing the invention, and do not restrict the scope of the invention. Particularly, in the drawings, some elements are slightly exaggerated for the purpose of easy understanding of the invention. Since the drawings are used to easily understand the invention, it should be noted that widths, thicknesses, and the like of elements illustrated in the drawings might change at the time of actual implementation thereof.

FIGS. 1, 2 and 3 exemplarily illustrate an intervertebral cage according to one embodiment of the present invention;

FIGS. 4, 5, 6, 7 and 8 exemplarily illustrate a first rotatable body of the intervertebral cage according to one embodiment of the present invention;

FIGS. 9, 10, 11, 12 and 13 exemplarily illustrate a second rotatable body of the intervertebral cage according to one embodiment of the present invention;

FIGS. 14, 15 and 16 exemplarily illustrate ball joints of the intervertebral cage according to one embodiment of the present invention;

FIGS. 17, 18, 19 and 20 exemplarily illustrate an intervertebral cage according to another embodiment of the present invention; and

FIG. 21 exemplarily illustrates a process of inserting the intervertebral cage between vertebrae according to an embodiment of the present invention.

### DESCRIPTION OF EMBODIMENTS

Embodiments which will be described below with reference to the accompanying drawings can be implemented singly or in combination with other embodiments. But this is not intended to limit the present invention to a certain embodiment, and it should be understood that all changes, modifications, equivalents or replacements within the spirits and scope of the present invention are included. Especially, any of functions, features, and/or embodiments can be implemented independently or jointly with other embodiments. Accordingly, it should be noted that the scope of the invention is not limited to the embodiments illustrated in the accompanying drawings.

On the other hand, among terms used in this specification, terms such as “substantially,” “almost,” and “about” are used to take consideration of a margin or an error at the time of actual embodiment. For example, “substantially 90 degrees” should be construed to include angles at which the same advantages as at 90 degrees can be expected. For example, “almost zero” should be construed to include a quantity which is slightly present but is ignorable.

On the other hand, unless otherwise mentioned, “side” or “horizontal” is used to mention a right-left direction in the drawings, and “vertical” is used to mention an up-down direction in the drawings. Unless otherwise defined, an angle, an incidence angle, and the like are defined with respect to a virtual straight line perpendicular to a horizontally flat surface illustrated in the drawings.

Throughout the accompanying drawings, the same or similar elements are referred to using the same reference numerals.

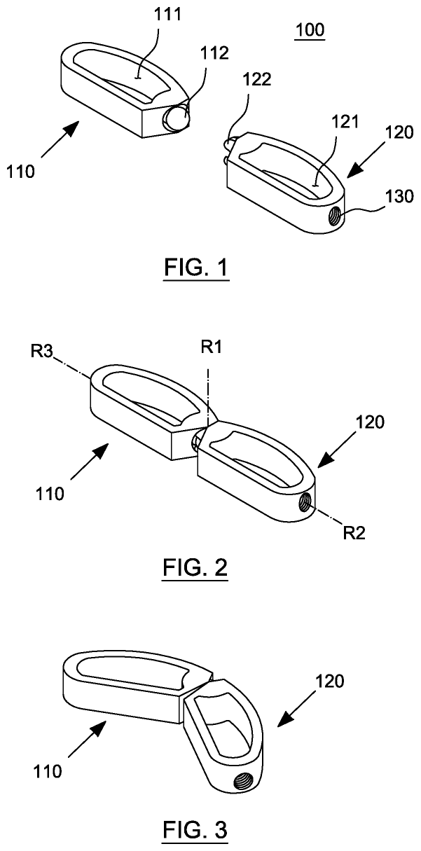

FIG. 1 exemplarily illustrates an exploded perspective view of an intervertebral cage according to one embodiment of the present invention, FIG. 2 exemplarily illustrates a perspective view showing an intervertebral cage aligned in parallel for insertion into the body, and FIG. 3 exemplarily illustrates a perspective view showing the intervertebral cage rotated in an arc shape after being inserted into the body.

The intervertebral cage 100 may include a first rotatable body 110, a second rotatable body 120, and ball joints 112, 122. The first rotatable body 110, the second rotatable body 120, and the ball joints 112, 122 may be formed of a resin such as polyetheretherketone (PEEK) or a metal such as titanium. Meanwhile, the first rotatable body 110 and the second rotatable body 120 may have substantially the same thickness. The first rotatable body 110 and the second rotatable body 120 may have substantially the same shape. At least one of the first rotatable body 110 and the second rotatable body 120 may include bone powder accommodating holes 111, 121 for accommodating bone powder therein. On the right side of the second rotatable body 120, a fastening groove 130 screwed with an insertion rod (not shown) for inserting the intervertebral cage 100 into the body may be formed.

The first rotatable body 110 may be rotatably coupled to a lateral surface of the second rotatable body 120 by ball joints 112, 122, and thus, the second rotatable body 130 may be also rotatably coupled to a lateral surface of the first rotatable body 110. When being rotatably coupled to the ball joints 112, 122, the first rotatable body 110 or the second rotatable body 120 may rotate in a counterclockwise direction (when the first rotatable body 110 rotates) or clockwise direction (when the second rotatable body 120 rotates) around axis R1.

Referring to FIG. 2, the first rotatable body 110 and the second rotatable body 120 may rotate in a lengthwise direction of each body. The first rotatable body 110 and the second rotatable body 120 may be perpendicular to the axis R1 by the ball joints 112, 122 and rotate in the clockwise or counterclockwise direction around axes R2 and R3 extending in the lengthwise direction of the respective bodies 110, 120. In a state where the first rotatable body 110 and the second rotatable body 120 are aligned in parallel, rotation of the first rotatable body 110 and the second rotatable body 120 about axes R2 and R3 is not restricted. However, in a state where the first rotatable body 110 and the second rotatable body 120 are rotated around the axis R1, the rotation of the first rotatable body 110 and the second rotatable body 120 around the axes R2 and R3 may be restricted.

Referring to FIG. 3, the intervertebral cage 100 inserted between vertebrae may have an arc shape. From a view of the second rotatable body 120, the first rotatable body 110 may rotate in a direction in which the lateral surface of the first rotatable body 110 faces the lateral surface of the second rotatable body 120, for example, in a counterclockwise direction around axis R1. A top surface of the first rotatable body 110 and a top surface of the second rotatable body 120 may be curved or flat when viewed from the front. When the first rotatable body 110 rotates maximally, the top surface of the intervertebral cage 100 may have an arc shape extending from a left end of the first rotatable body 110 to a right end of the second rotatable body 120. In one embodiment, the lateral surface 116 of the first rotatable body 110 and the lateral surface 126 of the second rotatable body 120 may contact each other. In this case, when the first rotatable body 110 rotates about the axis R3, an angle between the first rotatable body 110 and the second rotatable body 120 may increase. In another embodiment, the lateral surface 116 of the first rotatable body 110 and the lateral surface 126 of the second rotatable body 120 may not contact each other. In this case, when the first rotatable body 110 rotates around the axis R3, the angle between the first rotatable body 110 and the second rotatable body 120 may be maintained.

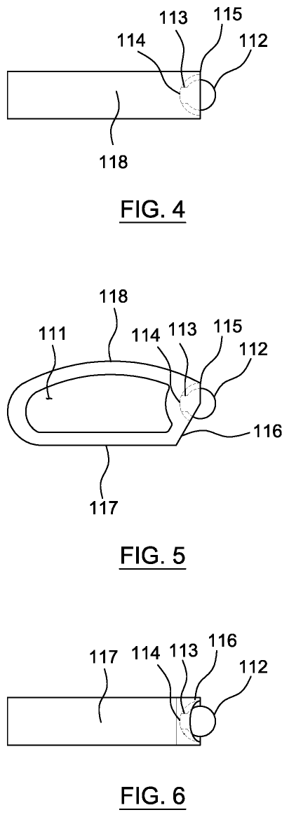

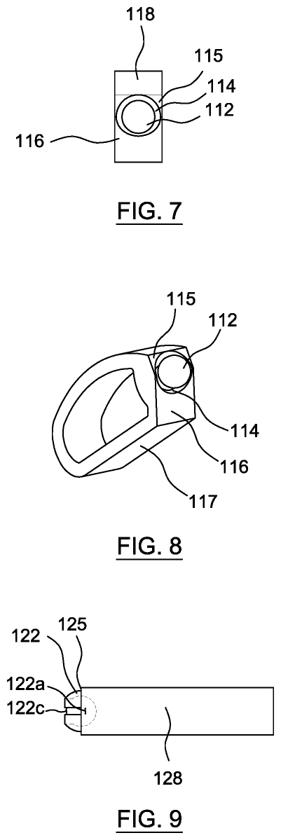

FIGS. 4 through 8 exemplarily illustrate a first rotatable body of the intervertebral cage according to one embodiment of the present invention, FIG. 4 exemplarily illustrates a top view of the first rotatable body, FIG. 5 exemplarily illustrates a front view of the first rotatable body, FIG. 6 exemplarily illustrates a bottom view of the first rotatable body, FIG. 7 exemplarily illustrates a right view of the first rotatable body, and FIG. 8 exemplarily illustrates a left view of the first rotatable body.

Referring FIGS. 4 to 8 together, the first rotatable body 110 may include a bone powder receiving hole 111 configured for penetrating the inside and a ball 112. A bottom surface 117 of the first rotatable body 110 may be substantially flat, the top surface 118 may be a convex curved surface, and the front and rear surfaces of the first rotatable body 110 may be substantially flat. In one embodiment, the lateral surfaces 115 and 116 may be flat. In a case that the lateral surfaces 115, 116 are flat, the first lateral surface 115 may extend from the top surface 118 toward the bottom surface 117, and the second lateral surface 116 extending from the first lateral surface 115 to the bottom surface 117 may be inclined. In another embodiment, the lateral surfaces 115 and 116 may be convex curved surfaces when viewed from the front.

The spherical ball 112 may be disposed on the lateral surfaces 115, 116 of the first rotatable body 110. A connecting rod 113 connects the ball 112 to the first rotatable body 110. The connecting rod 113 may extend from the lateral surfaces 115, 116 to the ball 112 along the lengthwise direction of the first rotatable body 110. In the illustrated structure, some of the lateral surfaces 115, 116 may be recessed into the first rotatable body 110. The recessed lateral surface 114 may be a curved surface, and a space formed between the ball 112 and the connecting rod 113 by the recessed lateral surface 114 may accommodate a part of the ball housing 122.

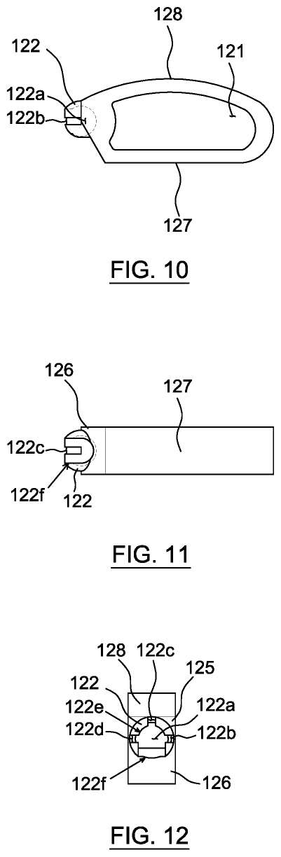

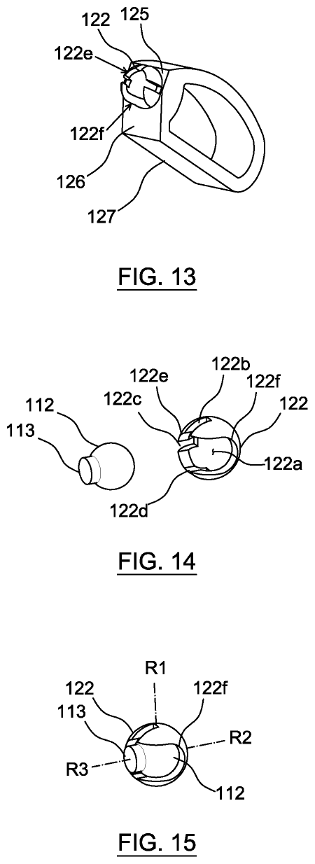

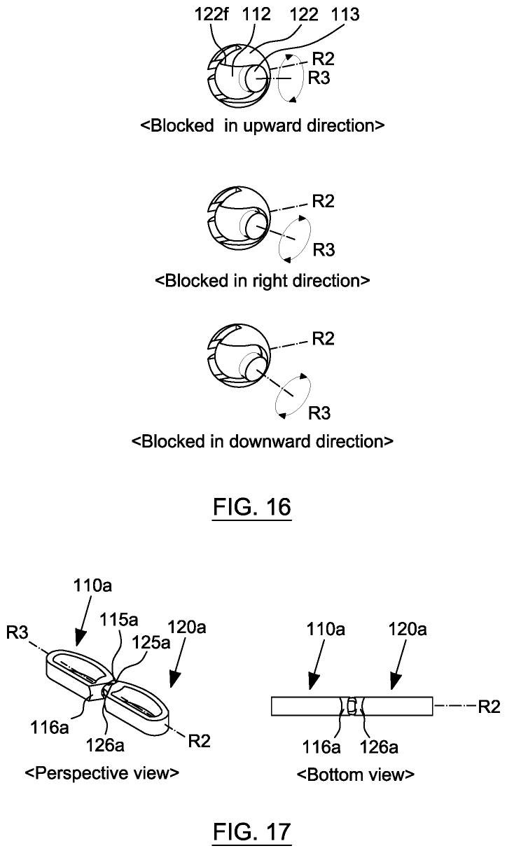

FIGS. 9, 10, 11, 12 and 13 exemplarily illustrate a second rotatable body of the intervertebral cage according to one embodiment of the present invention, FIG. 9 exemplarily illustrates a top view of the second rotatable body, FIG. 10 exemplarily illustrates a front view of the second rotatable body, FIG. 11 exemplarily illustrates a bottom view of the second rotatable body, FIG. 12 exemplarily illustrates a right view of the second rotatable body, and FIG. 13 exemplarily illustrates a left view of the second rotatable body.

Referring together with FIGS. 9 to 13, the second rotatable body 120 may include a ball housing having a bone powder accommodating hole 121 configured for penetrating the inside. The bottom surface 127 of the second rotatable body 120 may be substantially flat, the top surface 128 may be the convex curved surface, and the front and rear surfaces of the second rotatable body 120 may be substantially flat. In one embodiment, the lateral surfaces 125, 126 may be flat. When the lateral surfaces 125, 126 are flat, the first lateral surface 125 may extend from the top surface 128 toward the bottom surface 127, and the second lateral surface 126 extending from the first lateral surface 125 to the bottom surface 127 may be inclined. In another embodiment, the lateral surfaces 125, 126 may be convex curved surfaces when viewed from the front.

The ball housing 122 may be disposed on the lateral surfaces 125, 126 of the second rotatable body 120. The ball housing 122 may accommodate the ball 112 and define a rotation path along which the connecting rod 113 moves when the ball 112 rotates around the axis R1. In the illustrated structure, the ball housing 122 may be a sphere having a ball accommodating space for accommodating the ball 112 therein. At least a portion of the ball housing 122 may be integrally formed with the second rotatable body 120. Due to this, a portion of the ball accommodating space may be formed inside the second rotatable body 120.

A ball insertion hole 122e smaller than a diameter of the ball 112 may be formed on the surface of the ball housing 122 facing the ball 112. The ball insertion hole 122e may be formed to extend from the left lateral surface of the ball housing 122 to the ball accommodating space 112a. A plurality of elastic grooves 122b, 122c, and 122d for expanding the diameter of the ball insertion hole 122e when the ball 112 is inserted may be formed to extend from the ball insertion hole 122e toward the lateral surfaces 125, 126 of the second rotatable body 120. A guide groove 122f may be formed to extend from the ball insertion hole 122e toward the lateral surface 126 of the second rotatable body 120. The guide groove 122f becomes the rotation path along which the connecting rod 113 can move when the first rotatable body 110 connected to the ball 112 by the connecting rod 113 rotates around the axis R1.

FIGS. 14, 15 and 16 exemplarily illustrate ball joints of the intervertebral cage according to one embodiment of the present invention.

FIG. 14 exemplarily illustrates a way of coupling the ball 112 to the ball housing 122. When the ball 112 is pushed toward the ball insertion hole 122e, a width of one end of the plurality of elastic grooves 122b, 122c, and 122d, that is, a portion connected to the ball insertion hole 122e may be expanded, so that the diameter of the ball insertion hole 122e increases. Then, when the ball 112 is pushed to the ball accommodating space, the extended ends of the plurality of elastic grooves 122b, 122c, and 122d return to their original states.

FIG. 15 exemplarily illustrates ball joints 112, 122 in a state in which the first rotatable body 110 and the second rotatable body 120 are aligned in parallel. In this state, the ball 112 and the connecting rod 113 may rotate around the axis R3, and the ball housing 122 may rotate around the axis R2. In addition, since the width (or diameter) of the connecting rod 113 is smaller than that of the ball insertion hole 122e, the ball 112 and the connecting rod 113 may finely rotate in a vertical direction within a range where the connecting rod 113 is not limited by the ball insertion hole 122e. However, in the structures illustrated in FIGS. 1 to 13, the lateral surface 115 of the first rotatable body 110 and the lateral surface 125 of the second rotatable body 120 may contact each other while aligned in parallel. Due to this, the rotation of the first rotatable body 110 in the vertical direction may be restricted.

FIG. 16 exemplarily illustrates the ball joints 112, 122 in a state in which the first rotatable body 110 rotates to the maximum. In this state, the ball 112 and the connecting rod 113 can rotate about the axis R3, but the lateral surface 116 of the first rotatable body 110 and the lateral surface 126 of the second rotatable body 120 may restrict the rotation of the ball 112 and the connecting rod 113. In one embodiment, if there is an angle between the lateral surface 116 and the lateral surface 126, the ball 112 and the connecting rod 113 may rotate about the axis R3 until the lateral surface 116 and the lateral surface 126 contact each other. In another embodiment, if the lateral surface 116 and the lateral surface are in contact with each other, the rotation of the ball 112 and the connecting rod 113 may widen the angle between the first rotatable body 110 and the second rotatable body 120. In addition, the rotation of the ball housing 122 around the axis R2 may be blocked. If a width (or diameter) of the guide groove 122f is sufficiently larger than the width of the connecting rod 113, the ball housing 122 may rotate finely up and down within a range where the connecting rod 113 is not blocked by the guide groove 122f.

The length of the guide groove 122f, for example, a distance from a vicinity of the ball insertion groove 122e (namely, one end of the guide groove 122f) to the vicinity of the lateral surface 126 (namely, the other end of the guide groove 122f) along the lengthwise direction of the second rotatable body 120 may define the maximum rotation angle of the first rotatable body 110. Meanwhile, the width of the guide groove 122f may be constant or may increase near the other end of the guide groove 122f. For example, the guide groove 122f may decrease from the vicinity of one end to the vicinity of the other end and then increase at the other end.

FIGS. 17, 18, 19 and 20 exemplarily illustrate an intervertebral cage according to another embodiment of the present invention. Compared to the embodiment illustrated in FIGS. 1 to 13, in the intervertebral cage illustrated in FIGS. 17 to 20, the lateral surfaces 115a, 116a of the first rotatable body 110a and the lateral surfaces 125a, 126a of the second rotatable body 120a are formed as a convex curved surface when viewed from the bottom.

FIG. 17 exemplarily illustrates a state in which the first rotatable body 110a and the second rotatable body 120a are aligned in parallel. In this state, the lateral surface 115a of the first rotatable body 110a and the lateral surface 125a of the second rotatable body 120a may contact each other. Compared to the embodiment where the lateral surface 115 of the first rotatable body 110 and the lateral surface 125 of the second rotatable body 120 are flat, the counterclockwise rotation of the first rotatable body 110a is restricted around the axis R1, but a fine rotation of the first rotating body 110a in the vertical direction may become possible.

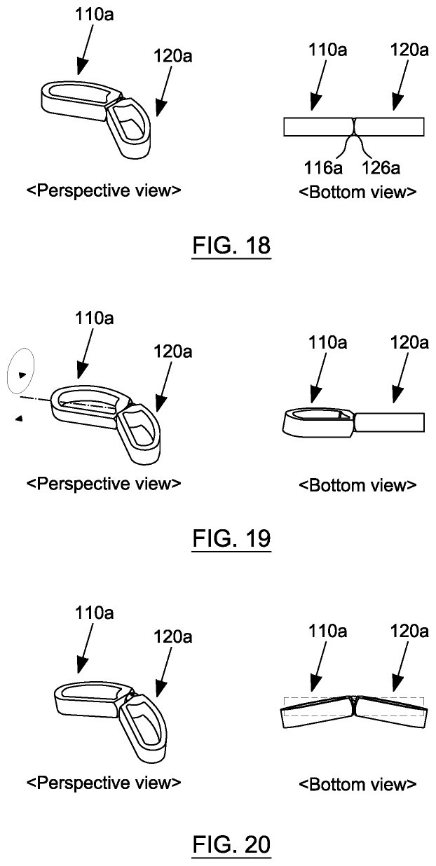

FIG. 18 exemplarily illustrates a state in which the first rotatable body 110a is rotated to the maximum. In this state, the lateral surface 116a of the first rotatable body 110a and the lateral surface 126a of the second rotatable body 120a may contact each other. The contact area between the lateral surface 116a and the lateral surface 126a becomes almost straight line.

FIG. 19 exemplarily illustrates a state in which the first rotatable body 110a is rotated about the axis R3, and FIG. 20 shows a state in which the first rotatable body 110a and the second rotatable body 120a are slightly rotated in a downward direction. The lateral surface 116a and the lateral surface 126a, which are convex surfaces, may not push each other when the first rotatable body 110a and the second rotatable body 120a rotate finely in the downward direction. Therefore, during the fine rotation, the angle between the first rotatable body 110a and the second rotatable body 120a may not change.

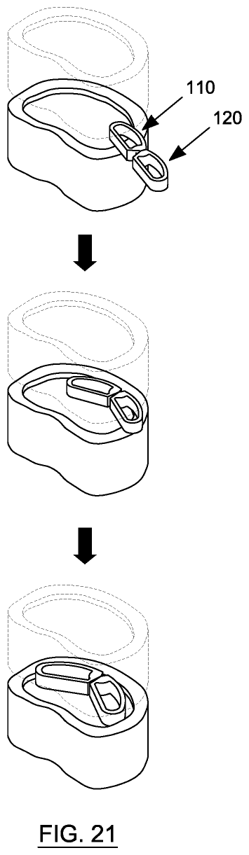

FIG. 21 exemplarily illustrates a process of inserting the intervertebral cage between vertebrae according to an embodiment of the present invention.

A minimum incision is made on the patient's back, and the intervertebral cage 100 aligned in parallel is inserted through the incision. To make room for the intervertebral cage 100 to be seated, the discs between the vertebrae may be removed. The intervertebral cage 100 may be inserted while the bone fragments are accommodated in the bone fragment receiving holes 111, 121.

When the intervertebral cage 100 is pushed in the initial insertion direction, a distal end of the first rotatable body 110 touches a medial wall of the vertebrae, causing the first rotatable body 110 to rotate counterclockwise toward the second rotatable body 120. Thus, the intervertebral cage 100 may become the arc shape. Continuing to push the intervertebral cage 100 in the initial insertion direction, the first rotatable body 110 will reach the opposite side of the incision along the medial wall of the vertebrae.

The fully inserted intervertebral cage 100 may sit on the inside of the medial wall of the vertebrae.

The aforementioned description for the present invention is exemplary, and those skilled in the art can understand that the invention can be modified in other forms without changing the technical concept or the essential feature of the invention. Therefore, it should be understood that the embodiments described above are illustrative and non-limiting in all respects. In particular, the features of the present invention described with reference to the drawings are not limited to the structures shown in the specific drawings, and may be implemented independently or in combination with other features.

The scope of the invention is defined by the appended claims, not by the above detailed description, and it should be construed that all changes or modifications derived from the meanings and scope of the claims and equivalent concepts thereof are included in the scope of the invention.

What is claimed is:

1. An intervertebral cage, comprising:
a first rotatable body;
a second rotatable body; and
ball joints, disposed on a lateral surface of the first rotatable body and a lateral surface of the second rotatable body facing the first rotatable body, and configured for coupling the first rotatable body rotatably to the second rotatable body and defining a rotation path through which the first rotatable body rotates toward the second rotatable body.

2. The intervertebral cage of claim 1, wherein the ball joints comprises
a ball;
a connecting rod, configured for connecting the ball and the lateral surface of the first rotatable body; and
a ball housing, coupled to the lateral surface of the second rotatable body and configured for accommodating the ball,
wherein the ball housing comprises
a ball accommodating space, configured for accommodating the ball,
a ball insertion hole, formed to extend from a surface of the ball housing facing the lateral surface of the second rotatable body to the ball accommodation space and having a smaller diameter than that of the ball,
a plurality of elastic grooves, extending from the ball insertion hole toward the lateral surface of the second rotatable body,
and
a guide groove, extending from the ball insertion hole toward the lateral surface of the second rotatable body to define the rotation path along which the connecting rod moves when the ball rotates about its center as an axis.

3. The intervertebral cage of claim 2, wherein at least a portion of the ball accommodating space is formed to extend inwardly from the lateral surface of the second rotatable body.

4. The intervertebral cage of claim 1, wherein a bottom surface of each of the first rotatable body and the second rotatable body is flat,
wherein the bottom surfaces of the first rotatable body, and the second rotatable body are arranged in parallel before the first rotatable body and the second rotatable body rotate.

5. The intervertebral cage of claim 1, wherein a top surface of each of the first rotatable body and the second rotatable body is a curved surface,
wherein top surfaces of the first rotatable body and the second rotatable body are arranged in an arc shape when the first rotating body rotates.

6. The intervertebral cage of claim 1, wherein the lateral surface of the first rotatable body and the lateral surface of the second rotatable body are convex curved surfaces.

7. The intervertebral cage of claim 1, wherein the lateral surface of the first rotatable body and the lateral surface of the second rotatable body are inclined with respect to the respective bottom surfaces.

8. The intervertebral cage of claim 1, wherein at least one of the first rotatable body and the second rotatable body comprises a bone powder receiving hole penetrating the inside.
